(19) United States
(12) Patent Application Publication
(10) Pub. No.: US 2011/0313448 A1
(43) Pub. Date: Dec. 22, 2011

(54) Wall mounted back massage "RINO"

(76) Inventor: Allison Patricia Wong, Weston, FL (US)

(21) Appl. No.: 12/802,914

(22) Filed: Jun. 17, 2010

(51) Int. Cl. A61B 17/00

(52) U.S. Cl. 606/204

(57) ABSTRACT

RINO's is a wall mounted back massaging apparatus. Its designed with two-protruding digits that extend from a flat base. Spaced at an angle of sixty degrees to safely apply relief to pressure points on both sides of the spinal cord at once. Attachable to a wall with the use of Velcro. Unibody construction for durability. The idea is to be able to self erase tension in the muscles in your back. The amount of pressure depends on the user's body weight and the angle in which placement of their feet are from the wall. (The greater the distance of the user's feet from the wall, the deeper the muscle tissue massage)

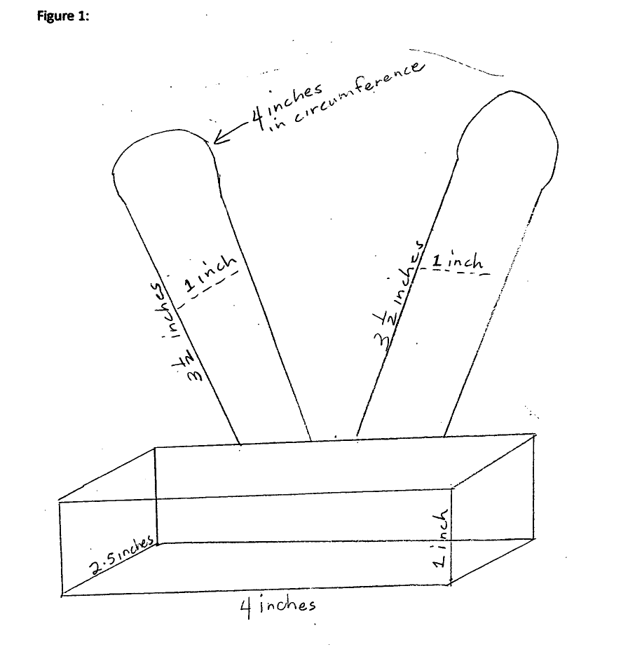

### SUMMARY

RINO is a; two protruding digit, adjustable wall mounted, uni-body constructed, personal body pressure point relief unit, It is intended for use to massage all pressure points in the body.

### SUMMARY DETAILS

Two-protruding digits are spaced at an angle of sixty degrees to safely apply relief to pressure points on both sides of the spinal cord at once. Intended to be attached to a wall by heavy duty Velcro. Velcro can be placed on several areas of the wall to enable mobility of the unit. Uni-body construction for sturdy and economical manufacturing. Construction of the prototype is of oven baked clay. The idea is to be able to rub out tension yourself instead of depending on someone else to do it for you, either a professional masseuse or a friend or partner. The user applies the amount of pressure in the way of body weight, by varying the distance and angle in which the placement of their feet from the wall. The further the distance from the wall the greater the angle and the more body weight resulting in a deeper muscle tissue massage. RINO's design has been tested and proven sturdy enough to withstand the weight of a male, two hundred and fifty pounds, six feet four inches in height.

### DRAWING

Drawing Description:

Base four inches long two and a half inches wide, one inch thick.

Each of two protruding digits one inch square shaft, three and a half inches in length, rounded tips four inches in circumference.

Heavy duty velcro is required to support the weight of RINO. The prototype constructed of baked clay weighs approximately four pounds.

### DRAWING DETAILED DESCRIPTION

Mold the base of four inches long by two and a half inches wide and one inch thick.

Form two one inch holes in the center of the base for the two-sixty degree angled digits.

Construct each digit as one inch square on each side and three and a half inches in length

The tip of each digit is to be rounded for easy glide against the skin.

Place each digit, to form a sixty degree angle once in place, mold the construction material around each digit to secure.

1. Each of two protruding digits is constructed one inch square and three and a half inches in length, sixty degrees apart and a rounded tip. This construction enables durability to withstand use by a person whose weight is two hundred and fifty pounds, and will not pierce the skin.
